(19) United States
(12) Patent Application Publication
(10) Pub. No.: US 2016/0007962 A1
(43) Pub. Date: Jan. 14, 2016

(54) CONDUCTOR INTERFACE FOR MINIMALLY INVASIVE MEDICAL SENSOR ASSEMBLY AND ASSOCIATED DEVICES, SYSTEMS, AND METHODS

(71) Applicant: Koninklijke Philips N.V., Amsterdam (NL)

(72) Inventor: Thomas Esbeck, Carlsbad, CA (US)

(21) Appl. No.: 14/796,796

(22) Filed: Jul. 10, 2015

Related U.S. Application Data
(60) Provisional application No. 62/023,509, filed on Jul. 11, 2014.

(51) Int. Cl. A61B 8/00; A61B 8/12

(52) U.S. Cl. CPC A61B 8/4494; A61B 8/12; A61B 2562/12

(57) ABSTRACT

A minimally invasive medical sensor assembly with a conductor interface is provided. The medical sensor assembly includes a medical sensor element disposed at a distal portion, a control circuit electrically coupled to the medical sensor element, and a conductor interface disposed at a proximal portion. The conductor interface can include a plurality of channels and a removable member. In one embodiment, the medical sensor assembly is an ultrasound scanner assembly for an intravascular ultrasound (IVUS) device. An ultrasound scanner assembly and a method of manufacturing an IVUS device are also provided. Further, an IVUS device is provided. The IVUS device can include an ultrasound scanner assembly having a conductor interface disposed at a proximal portion. The IVUS device can include a cable having a connector at a distal portion. The connector can be configured to mechanically engage the conductor interface to electrically couple the ultrasound scanner assembly and another component.

### CROSS REFERENCE TO RELATED APPLICATIONS

The present application claims priority to and the benefit of the U.S. Provisional Patent Application No. 62/023,509, filed Jul. 11, 2014, which is hereby incorporated by reference in its entirety.

### TECHNICAL FIELD

The present disclosure relates generally to medical sensing devices including a conductor interface that facilitates electrical coupling between a minimally invasive medical sensing assembly and a cable extending along the length of the medical sensing device.

### BACKGROUND

Minimally invasive sensing systems are routinely utilized by medical professionals to evaluate, measure, and diagnose conditions within the human body. As one example, intravascular ultrasound (IVUS) imaging is widely used in interventional cardiology as a diagnostic tool for assessing a diseased vessel, such as an artery, within the human body to determine the need for treatment, to guide the intervention, and/or to assess its effectiveness. An IVUS device includes one or more ultrasound transducers arranged at a distal end of an elongate member. The elongate member is passed into the vessel thereby guiding the transducers to the area to be imaged. The transducers emit ultrasonic energy in order to create an image of the vessel of interest. Ultrasonic waves are partially reflected by discontinuities arising from tissue structures (such as the various layers of the vessel wall), red blood cells, and other features of interest. Echoes from the reflected waves are received by the transducer and passed along to an IVUS imaging system. The imaging system processes the received ultrasound echoes to produce a cross-sectional image of the vessel where the device is placed.

There are two general types of IVUS devices in use today: rotational and solid-state (also known as synthetic aperture phased array). For a typical rotational IVUS device, a single ultrasound transducer element is located at the tip of a flexible driveshaft that spins inside a plastic sheath inserted into the vessel of interest. The transducer element is oriented such that the ultrasound beam propagates generally perpendicular to the axis of the device. The fluid-filled sheath protects the vessel tissue from the spinning transducer and driveshaft while permitting ultrasound signals to propagate from the transducer into the tissue and back. As the driveshaft rotates, the transducer is periodically excited with a high voltage pulse to emit a short burst of ultrasound. The same transducer then listens for the returning echoes reflected from various tissue structures. The IVUS imaging system assembles a two dimensional display of the vessel cross-section from a sequence of pulse/acquisition cycles occurring during a single revolution of the transducer.

In contrast, solid-state IVUS devices utilize a scanner assembly that includes an array of ultrasound transducers distributed around the circumference of the device connected to a set of transducer controllers. The transducer controllers select transducer sets for transmitting an ultrasound pulse and for receiving the echo signal. By stepping through a sequence of transmit-receive sets, the solid-state IVUS system can synthesize the effect of a mechanically scanned transducer element but without moving parts. Since there is no rotating mechanical element, the transducer array can be placed in direct contact with the blood and vessel tissue with minimal risk of vessel trauma. Furthermore, because there is no rotating element, the interface is simplified. The solid-state scanner can be wired directly to the imaging system with a simple electrical cable.

Conventionally, conductors of the electrical cable are individually soldered to an ultrasound scanner assembly during manufacture of the solid-state IVUS device. The particular locations on the ultrasound scanner assembly where the individual conductors are soldered are very small. Furthermore, the conductors must be connected to the ultrasound scanner assembly so that there is no inadvertent electrical signal flow between the conductors. For example, excess or misplaced solder can cause errant signal flow. Thus, establishing proper electrical communication is highly labor intensive and time consuming process. Moreover, the strength of the connection varies based on each particular solder joint, which adds uncertainty to the reliability of connection. The conventional soldering process also generates a relatively higher amount of scrap, which is economically inefficient.

While existing medical sensing systems have proved useful, there remains a need for improvements in the design of the sensor assembly to reduce the time and labor involved in coupling the electrical cable and the sensor assembly, to improve reliability of the resulting connection, and to increase the efficiency of the manufacturing process. Accordingly, the need exists for improvements to the sensor assembly and its components, and to the methods used in manufacturing these elements.

### SUMMARY

Embodiments of the present disclosure provide an improved connection between a sensor assembly and an electrical cable in a medical sensing device. In some embodiments, the sensor assembly includes a conductor interface. The conductor interface includes channels that receive individual conductors of the cable. The conductor interface also includes a removable tab that electrically isolates the individual conductors after they have been soldered in the conductor interface. Removing the tab removes excess solder from the conductor interface that can cause unintentional and unwanted electrical signal flow. In some embodiments, the cable includes a distal connector that houses all of the conductors. The distal connector can be mechanically coupled to the conductor interface of the sensor assembly. One example of the sensor assembly described herein is an ultrasound scanner assembly for intravascular ultrasound (IVUS) devices.

In an exemplary aspect, the present disclosure is directed to method of manufacturing an intravascular ultrasound (IVUS) device. The method includes acquiring an ultrasound scanner assembly, the ultrasound scanner assembly including: an ultrasound transducer array disposed at a distal portion of the ultrasound scanner assembly; a transducer control circuit electrically coupled to the ultrasound transducer array; and a conductor interface disposed at a proximal portion of the ultrasound scanner assembly, wherein the conductor interface includes a plurality of channels and a removable member disposed at least partially along a surface of the conductor interface; acquiring a cable including a plurality of conductors; introducing each of a plurality of conductors into a respective channel of the conductor interface; electrically coupling the cable and the ultrasound scanner assembly; and removing the removable member from the conductor interface to electrically isolate the conductors of the cable from one another.

In another exemplary aspect, the present disclosure is directed to an ultrasound scanner assembly. The ultrasound scanner assembly includes an ultrasound transducer array disposed at a distal portion of the ultrasound scanner assembly; a transducer control circuit electrically coupled to the ultrasound transducer array; and a conductor interface disposed at a proximal portion of the ultrasound scanner assembly, wherein the conductor interface includes a plurality of channels and a removable member disposed at least partially along a surface of the conductor interface.

In another exemplary aspect, the present disclosure is directed to an intravascular ultrasound (IVUS) device. The IVUS device includes a flexible elongate member; a patient interface module (PIM) coupler disposed at a proximal portion of the flexible elongate member; an ultrasound scanner assembly disposed at a distal portion of the flexible elongate member, the ultrasound scanner assembly including: an ultrasound transducer array disposed at a distal portion of the ultrasound scanner assembly; a transducer control circuit electrically coupled to the ultrasound transducer array; and a conductor interface disposed at a proximal portion; a cable disposed within and extending along a length of the flexible elongate member between the ultrasound scanner assembly and the interface coupler, the cable including a plurality of conductors, the cable further including a connector at a distal portion configured to mechanically engage the conductor interface to electrically couple the interface coupler and the ultrasound scanner assembly.

In another exemplary aspect, the present disclosure is directed to a minimally invasive medical sensor assembly. The medical sensor assembly includes a medical sensor element disposed at a distal portion of the medical sensor assembly; a control circuit electrically coupled to the medical sensor element; and a conductor interface disposed at a proximal portion of the medical sensor assembly, wherein the conductor interface includes a plurality of channels and a removable member disposed at least partially along a surface of the conductor interface.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic schematic view of an intravascular ultrasound (IVUS) imaging system according to aspects of the present disclosure.

FIG. 2 is a top view of a portion of an ultrasound scanner assembly according to aspects of the present disclosure.

FIG. 3a is a perspective view of a conductor interface of an ultrasound scanner assembly according to aspects of the present disclosure.

FIG. 3b is a perspective view of a conductor interface of an ultrasound scanner assembly, similar to FIG. 3a, but including a removable member according to aspects of the present disclosure.

FIG. 4a is a diagrammatic top view of a conductor interface of an ultrasound scanner assembly according to aspects of the present disclosure.

FIG. 4b is a diagrammatic top view of a conductor interface of an ultrasound scanner assembly, similar to FIG. 4a, but including a removable member according to aspects of the present disclosure.

FIG. 5a is a cross-sectional front view of the conductor interface of the ultrasound scanner assembly of FIG. 4a.

FIG. 5b is a cross-sectional front view of the conductor interface of the ultrasound scanner assembly of FIG. 4b.

FIGS. 6-8 are cross-sectional front views of a conductor interface of an ultrasound scanner assembly during manufacturing an IVUS device according to aspects of the present disclosure.

FIG. 9a is a perspective view of a conductor interface of an ultrasound scanner assembly according to aspects of the present disclosure.

FIG. 9b is a perspective view of a conductor interface of an ultrasound scanner assembly, similar to FIG. 9a, but including a removable member according to aspects of the present disclosure.

FIG. 10a is a diagrammatic top view of a conductor interface of an ultrasound scanner assembly according to aspects of the present disclosure.

FIG. 10b is a diagrammatic top view of a conductor interface of an ultrasound scanner assembly, similar to FIG. 10a, but including a removable member according to aspects of the present disclosure.

FIG. 11a is a cross-sectional front view of the conductor interface of the ultrasound scanner assembly of FIG. 10a.

FIG. 11b is a cross-sectional front view of the conductor interface of the ultrasound scanner assembly of FIG. 10b.

FIG. 12a is a cross-sectional front view of the conductor interface of the ultrasound scanner assembly of FIG. 10a.

FIG. 12b is a cross-sectional front view of the conductor interface of the ultrasound scanner assembly of FIG. 10b.

FIG. 13a is a cross-sectional side view of the conductor interface of the ultrasound scanner assembly of FIG. 10a.

FIG. 13b is a cross-sectional side view of the conductor interface of the ultrasound scanner assembly of FIG. 10b.

FIGS. 14-16 are cross-sectional side views of a conductor interface of an ultrasound scanner assembly during manufacturing an IVUS device according to aspects of the present disclosure.

FIG. 17 is a flow diagram of a method of manufacturing an intravascular ultrasound (IVUS) device according to aspects of the present disclosure.

FIG. 18 is a top view of a conductor interface of an ultrasound scanner assembly and a distal connector of an electrical cable according to aspects of the present disclosure.

FIG. 19 is a side view of a distal connector of an electrical cable according to aspects of the present disclosure.

FIG. 20 is a cross-sectional front view of the distal connector of the cable of FIG. 18.

FIG. 21 is a cross-sectional side view of the conductor interface of the ultrasound scanner assembly of FIG. 18.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, while the minimally invasive medical sensing system is illustrated an IVUS system configured for cardiovascular imaging, it is understood that the sensing system is not intended to be limited to this application. The techniques and structures disclosed herein are equally adaptable for use in other medical sensing systems. Further, the IVUS system disclosed herein equally well suited to any application requiring imaging within a confined cavity. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

In some embodiments, the systems, devices, and methods of the present disclosure provide a conductor interface for an ultrasound scanner assembly. The conductor interface provides for more efficient electrical coupling between the ultrasound scanner assembly and conductors of an electrical cable extending along the IVUS device. The conductor interface includes one or more channels that receive individual conductors. Solder can be more freely and easily distributed along the channels to join the conductors to conductive pads within channels (compared to conventional methods of individually soldering conductors at specific locations of the ultrasound scanner assembly). The channels are physically separated such that the conductors are electrically isolated from one another. The conductor interface can further include a removable tab to further electrically isolate the conductors. While the solder is being more freely and easily distributed along the channels, any excess solder, which would otherwise create errant electrical signal flow paths, falls on the removable tab. The removable tab can be removed, along with the excess solder, to electrically isolate the conductors. In some embodiments, the systems, devices, and methods of the present disclosure provide a conductor interface for an ultrasound scanner assembly in combination with a distal connector for an electrical cable. The conductors of the cable can be received in the distal connector. The cable can be electrically coupled to the ultrasound scanner assembly when the distal connector mechanically engages the conductor interface. The embodiments of the present disclosure provide increased efficiency during manufacturing of the IVUS device by reducing the time and labor involved electrically coupling the conductors of the electrical cable and the ultrasound scanner assembly. The embodiments of the present disclose also improve reliability of the connection between the conductors and the ultrasound scanner assembly by providing connection methods that are less susceptible to variation between solder joints.

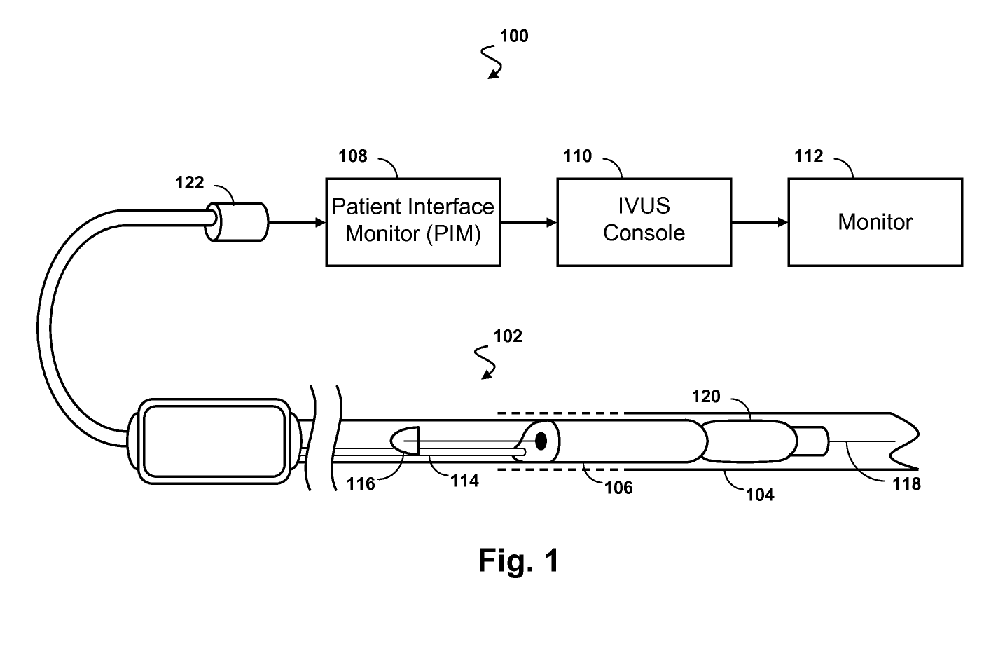

FIG. 1 is a diagrammatic schematic view of an ultrasound imaging system 100 according to an embodiment of the present disclosure. At a high level, an elongate member 102 (such as a catheter, guide wire, or guide catheter) of the imaging system 100 is advanced into a vessel 104. The distal-most end of the elongate member 102 includes a scanner assembly 106 with one or more ultrasound transducers and associated control circuitry. The ultrasound scanner assembly 106 is a non-limiting example of a medical sensor assembly. When the scanner assembly 106 is positioned near the area to be imaged, the ultrasound transducers are activated and ultrasonic energy is produced. A portion of the ultrasonic energy is reflected by the vessel 104 and the surrounding anatomy and received by the transducers. Corresponding echo information is passed along through a Patient Interface Monitor (PIM) 108 to an IVUS console 110, which renders the information as an image for display on a monitor 112.

The imaging system 100 may use any of a variety of ultrasonic imaging technologies. Accordingly, in some embodiments of the present disclosure, the IVUS imaging system 100 is a piezoelectric zirconate transducer (PZT) solid-state IVUS imaging system. In some embodiments, the system 100 incorporates capacitive micromachined ultrasonic transducers (cMUTs), and/or piezoelectric micromachined ultrasound transducers (PMUTs).

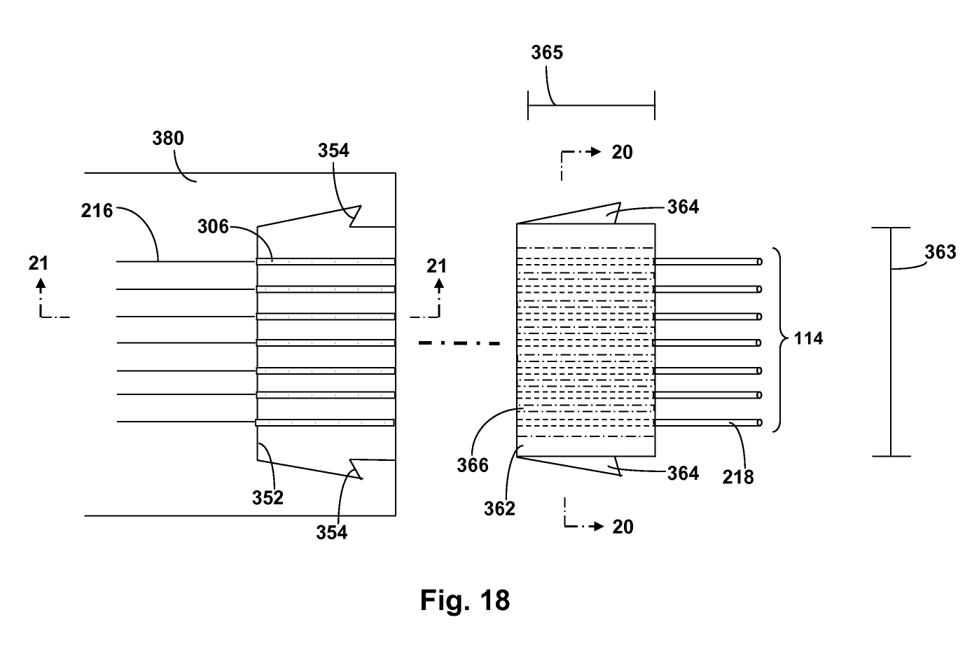

In some embodiments, the IVUS system 100 includes some features similar to traditional solid-state IVUS system, such as the EagleEye® catheter available from Volcano Corporation and those disclosed in U.S. Pat. No. 7,846,101 hereby incorporated by reference in its entirety. For example, the elongate member 102 includes the ultrasound scanner assembly 106 at a distal end of the member 102, which is coupled to the PIM 108 and the IVUS console 110 by a cable 114 extending along the longitudinal body of the member 102. The cable 114 caries control signals, echo data, and power between the scanner assembly 106 and the remainder of the IVUS system 100. The cable 114 can include a PIM coupler at a proximal portion. The cable 114 can include a plurality of conductors, including one, two, three, four, five, six, seven, or more conductors. It is understood that any suitable gauge wire can be used. In an embodiment, the cable 114 can include a seven-conductor transmission line arrangement utilizing, e.g., 44 AWG gauge wires. In an embodiment, the cable 114 can include a four-conductor transmission line arrangement with, e.g., 41 AWG gauge wires. In some embodiments, 43 AWG gauge wires can be used. In some embodiments, the cable 114 can include a distal connector (e.g., the distal connector 362 of FIG. 18) that is configured mechanically engaged to the scanner assembly 106. In some embodiments, the bare conductors of the cable 114 are electrically coupled to the scanner assembly 106.

In an embodiment, the elongate member 102 further includes a guide wire exit port 116. The guide wire exit port 116 allows a guide wire 118 to be inserted towards the distal end in order to direct the member 102 through a vascular structure (i.e., a vessel) 104. Accordingly, in some instances the IVUS device is a rapid-exchange catheter. In an embodiment, the elongate member 102 also includes an inflatable balloon portion 120 near the distal tip. The balloon portion 120 is open to a lumen that travels along the length of the IVUS device and ends in an inflation port (not shown). The balloon 120 may be selectively inflated and deflated via the inflation port.

The PIM 108 facilitates communication of signals between the IVUS console 110 and the elongate member 102 to control the operation of the scanner assembly 106. This includes generating control signals to configure the scanner, generating signals to trigger the transmitter circuits, and/or forwarding echo signals captured by the scanner assembly 106 to the IVUS console 110. With regard to the echo signals, the PIM 108 forwards the received signals and, in some embodiments, performs preliminary signal processing prior to transmitting the signals to the console 110. In examples of such embodiments, the PIM 108 performs amplification, filtering, and/or aggregating of the data. In an embodiment, the PIM 108 also supplies high- and low-voltage DC power to support operation of the circuitry within the scanner assembly 106.

The IVUS console 110 receives the echo data from the scanner assembly 106 by way of the PIM 108 and processes the data to create an image of the tissue surrounding the scanner assembly 106. The console 110 may also display the image on the monitor 112.

The ultrasound imaging system 100 may be utilized in a variety of applications and can be used to image vessels and structures within a living body. Vessel 104 represents fluid filled or surrounded structures, both natural and man-made, within a living body that may be imaged and can include for example, but without limitation, structures such as: organs including the liver, heart, kidneys, gall bladder, pancreas, lungs; ducts; intestines; nervous system structures including the brain, dural sac, spinal cord and peripheral nerves; the urinary tract; as well as valves within the blood or other systems of the body. In addition to imaging natural structures, the images may also include imaging man-made structures such as, but without limitation, heart valves, stents, shunts, filters and other devices positioned within the body.

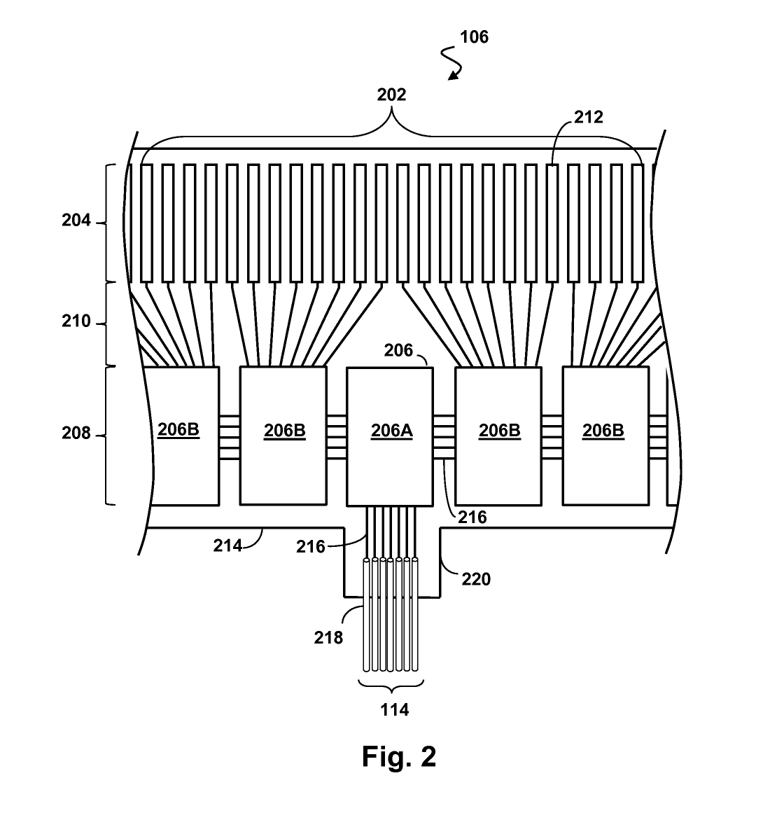

FIG. 2 is a top view of a portion of an ultrasound scanner assembly 106 according to an embodiment of the present disclosure. FIG. 2 depicts the ultrasound scanner assembly 106 in its flat form. The assembly 106 includes a transducer array 202 formed in a transducer region 204 and transducer control logic dies 206 (including dies 206A and 206B) formed in a control region 208, with a transition region 210 disposed therebetween. The transducer array 202 is a non-limiting example of a medical sensor element and/or a medical sensor element array. The transducer control logic dies 206 is a non-limiting example of a control circuit. The transducer region 204 is disposed adjacent a distal portion of the ultrasound scanner assembly. The control region 208 is disposed adjacent the proximal portion of the ultrasound scanner assembly. The transition region 204 is disposed between the control region 208 and the transducer region 204.

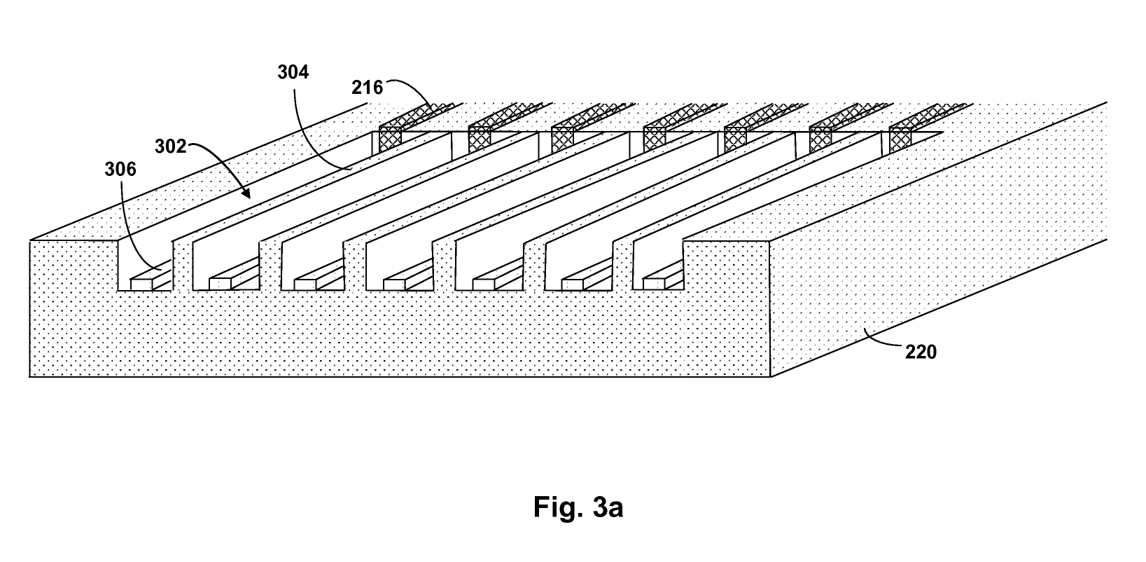

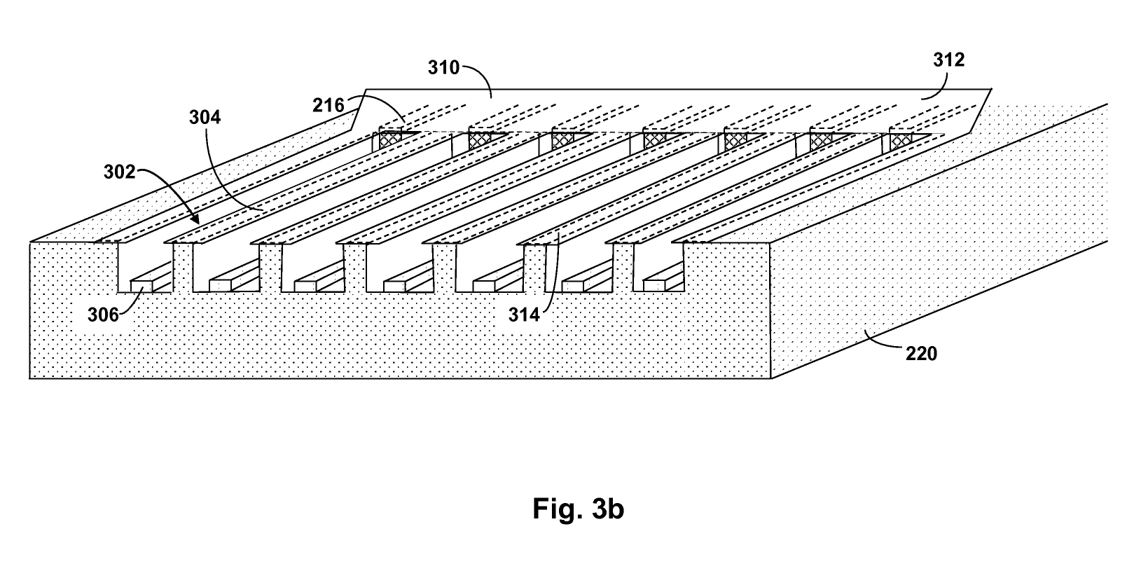

With respect to the transducer array 202, the array 202 may include any number and type of ultrasound transducers 212, although for clarity only a limited number of ultrasound transducers are illustrated in FIG. 3. In an embodiment, the transducer array 202 includes 64 individual ultrasound transducers 212. In a further embodiment, the transducer array 202 includes 32 ultrasound transducers 212. Other numbers are both contemplated and provided for. With respect to the types of transducers, in an embodiment, the ultrasound transducers 212 are piezoelectric micromachined ultrasound transducers (PMUTs) fabricated on a microelectromechanical system (MEMS) substrate using a polymer piezoelectric material, for example as disclosed in U.S. Pat. No. 6,641,540, which is hereby incorporated by reference in its entirety. In alternate embodiments, the transducer array includes piezoelectric zirconate transducers (PZT) transducers such as bulk PZT transducers, capacitive micromachined ultrasound transducers (cMUTs), single crystal piezoelectric materials, other suitable ultrasound transmitters and receivers, and/or combinations thereof.

The scanner assembly 106 may include various transducer control logic, which in the illustrated embodiment is divided into discrete control logic dies 206. In various examples, the control logic of the scanner assembly 106 performs: decoding control signals sent by the PIM 108 across the cable 114, driving one or more transducers 212 to emit an ultrasonic signal, selecting one or more transducers 212 to receive a reflected echo of the ultrasonic signal, amplifying a signal representing the received echo, and/or transmitting the signal to the PIM across the cable 114. In the illustrated embodiment, a scanner assembly 106 having 64 ultrasound transducers 212 divides the control logic across nine control logic dies 206, of which five are shown. Designs incorporating other numbers of control logic dies 206 including 8, 9, 16, 17 and more are utilized in other embodiments. In general, the control logic dies 206 are characterized by the number of transducers they are capable of driving, and exemplary control logic dies 206 drive 4, 8, and 16 transducers.

The control logic dies are not necessarily homogenous. In some embodiments, a single controller is designated a master control logic die 206A and contains the communication interface for the cable 114. Accordingly, the master control circuit may include control logic that decodes control signals received over the cable 114, transmits control responses over the cable 114, amplifies echo signals, and/or transmits the echo signals over the cable 114. The remaining controllers are slave controllers 206B. The slave controllers 206B may include control logic that drives a transducer 212 to emit an ultrasonic signal and selects a transducer 212 to receive an echo. In the depicted embodiment, the master controller 206A does not directly control any transducers 212. In other embodiments, the master controller 206A drives the same number of transducers 212 as the slave controllers 206B or drives a reduced set of transducers 212 as compared to the slave controllers 206B. In an exemplary embodiment, a single master controller 206A and eight slave controllers 206B are provided with eight transducers assigned to each slave controller 206B.

The transducer control logic dies 206 and the transducers 212 are mounted on a flex circuit 214 that provides structural support and interconnects for electrical coupling. The flex circuit 214 may be constructed to include a film layer of a flexible polyimide material such as KAPTON™ (trademark of DuPont). Other suitable materials include polyester films, polyimide films, polyethylene napthalate films, or polyetherimide films, other flexible printed semiconductor substrates as well as products such as Upilex® (registered trademark of Ube Industries) and TEFLON® (registered trademark of E.I. du Pont). The film layer is configured to be wrapped around a ferrule to form a cylindrical toroid in some instances. Therefore, the thickness of the film layer is generally related to the degree of curvature in the final assembled scanner assembly 106. In some embodiments, the film layer is between 5 μm and 100 μm, with some particular embodiments being between 12.7 μm and 25.1 μm.

To electrically interconnect the control logic dies 206 and the transducers, in an embodiment, the flex circuit 214 further includes conductive traces 216 formed on the film layer that carry signals between the control logic dies 206 and the transducers 212. The conductive traces 216 can also provide a set of conductive pads that contact the conductors 218 of cable 114 when the conductors are mechanically and electrically coupled to the flex circuit 214. In some embodiments, the flex circuit 214 can include conductive traces 216 only to serve as conductive pads to contact the conductors 218. In some embodiments, the flex circuit 214 can include conductive pads to contact the conductors 218 (e.g., conductive pads 306 of FIG. 3a) in addition to the conductive traces 216. Suitable materials for the conductive traces 216 and/or conductive pads include copper, gold, aluminum, silver, tantalum, nickel, and tin and may be deposited on the flex circuit 214 by processes such as sputtering, plating, and etching. In an embodiment, the flex circuit 214 includes a chromium adhesion layer. The width and thickness of the conductive traces 216 are selected to provide proper conductivity and resilience when the flex circuit 214 is rolled. In that regard, an exemplary range for the thickness of a conductive trace 216 and/or conductive pad is between 10-50 μm. For example, in an embodiment, 20 μm conductive traces 216 are separated by 20 μm of space. The width of a conductive trace 216 and/or a conductive pad on the flex circuit 214 may be further determined by the size of a pad of a device or the width of a wire to be coupled to the trace/pad.

A proximal portion of the flex circuit 214 can include a conductor interface 220. The conductor interface 220 can be a location of the flex circuit 214 where the conductors 218 of the cable 114 are coupled to the flex circuit. As described in greater detail herein, the conductor interface 220 and/or the conductor(s) 218 can be variously configured to efficiently couple the conductors 218 and the flex circuit 214. For example, the flex circuit 214 can include one or more features of conductor interface 220, conductor interface 370, and/or conductor interface 380, as described herein. In some embodiments, the substrate forming the conductor interface 220 is made of the same material(s) and/or is similarly flexible as the flex circuit 214. In other embodiments, the conductor interface 220 is made of different materials and/or is comparatively more rigid than the flex circuit 214. For example, the conductor interface 220 can be made of a plastic, thermoplastic, polymer, hard polymer, etc., including polyoxymethylene (e.g., DELRIN®), polyether ether ketone (PEEK), nylon, and/or other suitable materials.]

In some instances, the scanner assembly 106 is transitioned from a flat configuration to a rolled or more cylindrical configuration. For example, in some embodiments, techniques are utilized as disclosed in one or more of U.S. Pat. No. 6,776,763, titled “ULTRASONIC TRANSDUCER ARRAY AND METHOD OF MANUFACTURING THE SAME” and U.S. Pat. No. 7,226,417, titled “HIGH RESOLUTION INTRAVASCULAR ULTRASOUND TRANSDUCER ASSEMBLY HAVING A FLEXIBLE SUBSTRATE,” each of which is hereby incorporated by reference in its entirety.

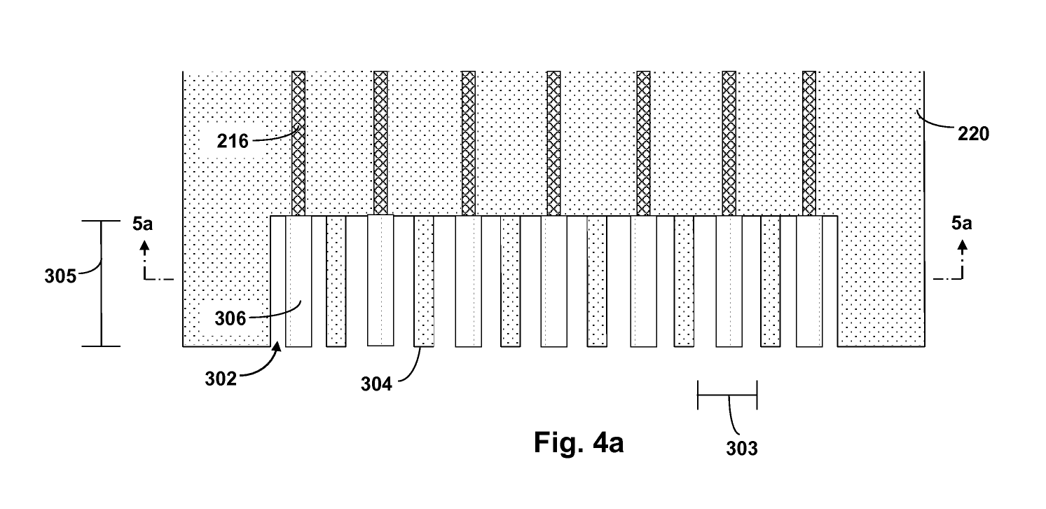

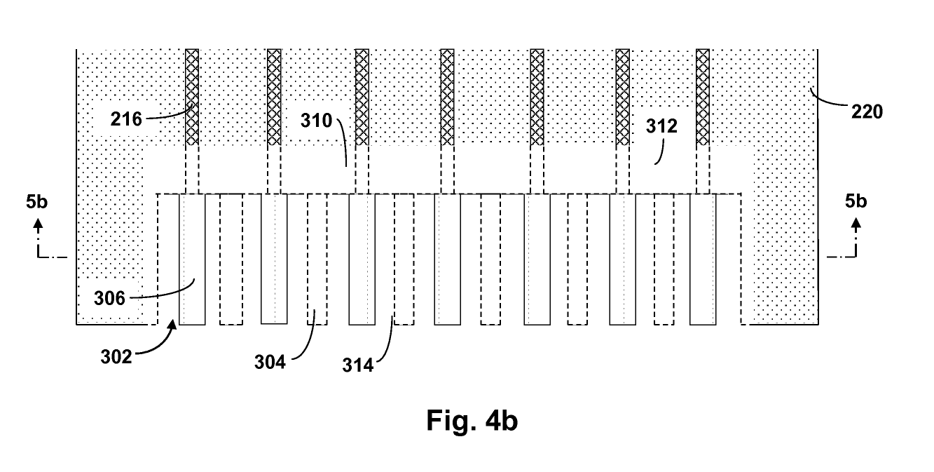

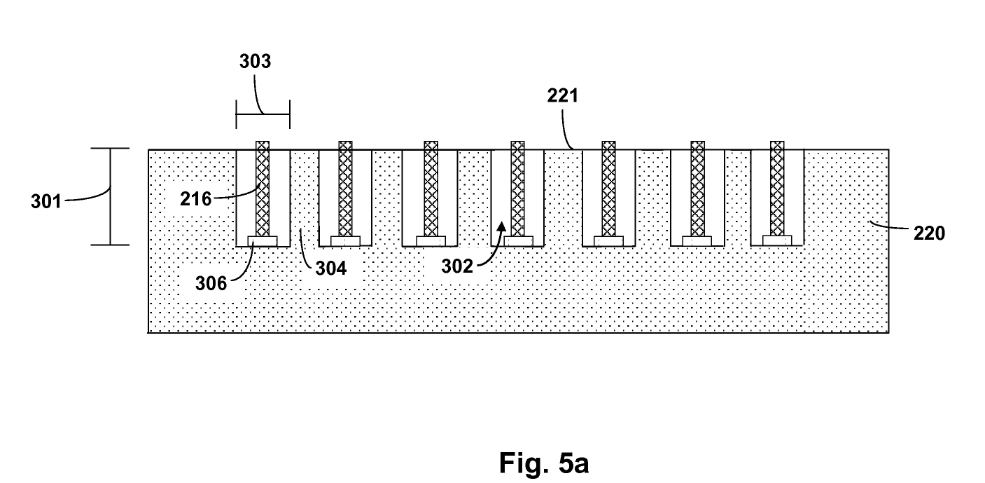

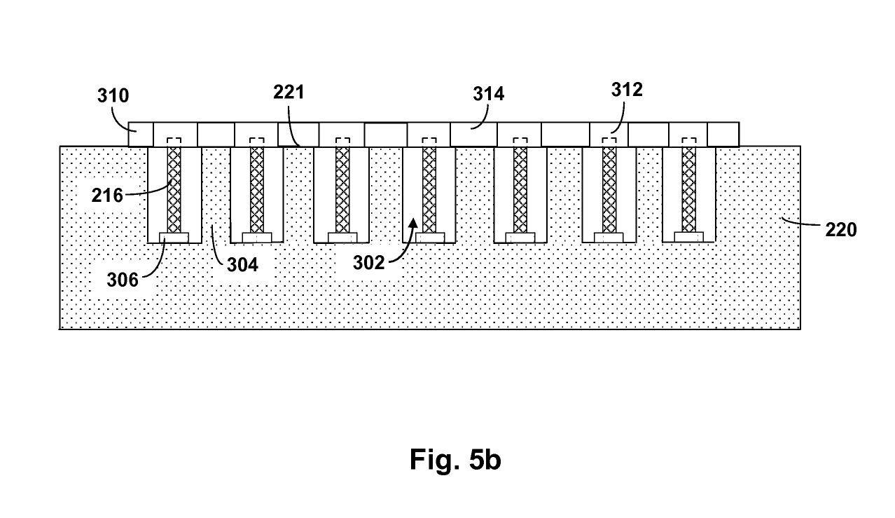

FIGS. 3a-5b illustrate an exemplary embodiment of the conductor interface 220 according to the present disclosure. FIGS. 3a and 3b are perspective views of the conductor interface 220. FIGS. 4a and 4b are top views of the conductor interface 220. FIGS. 5a and 5b are cross-sectional front views of the conductor interface 220 along the section lines indicated in FIGS. 4a and 4b, respectively. The conductor interface 220 of FIGS. 3b, 4b, and 5b includes a removable member 310, while the removable member 310 is omitted in the FIGS. 3a, 4a, and 5a.

Referring to FIGS. 3a, 4a, and 5a, the conductor interface 220 includes one or more channels 302. The channels 302 can be formed within conductor interface 220. For example, the channels 302 can be formed by removing material from the conductor interface 220, such as by mechanical and/or chemical etching. The channels 302 can be defined by walls 304 that are disposed between the one or more channels 302. The walls 304 can be portions of the conductor interface 220 that remain after material from the conductor interface 220 is removed to form the one or more channels 302. For example, the channels 302 can be formed by adding material to the conductor interface 220, such as by sputtering and/or plating. The walls 304 can be portions of material added to the conductor interface 220.

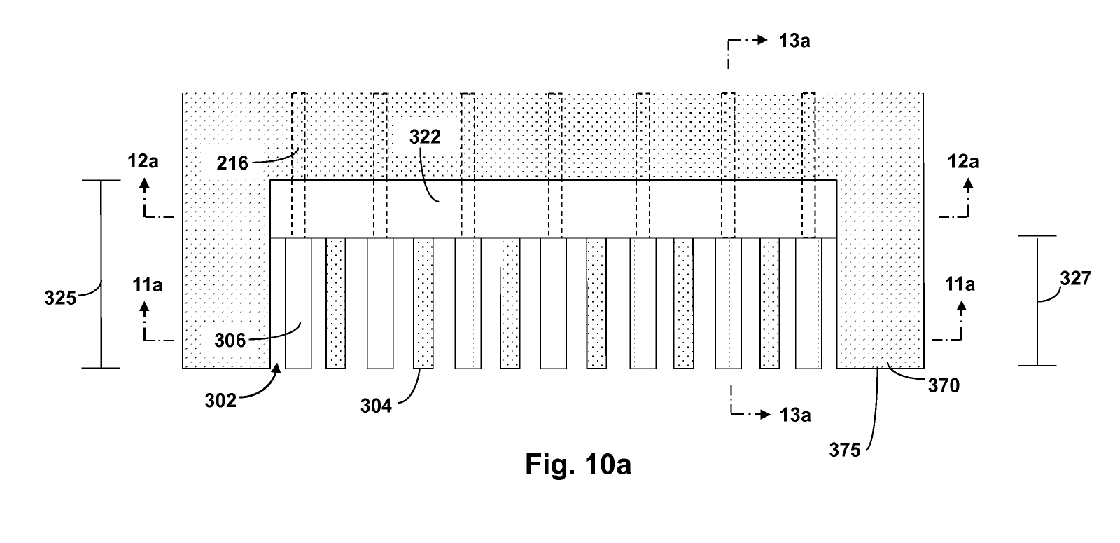

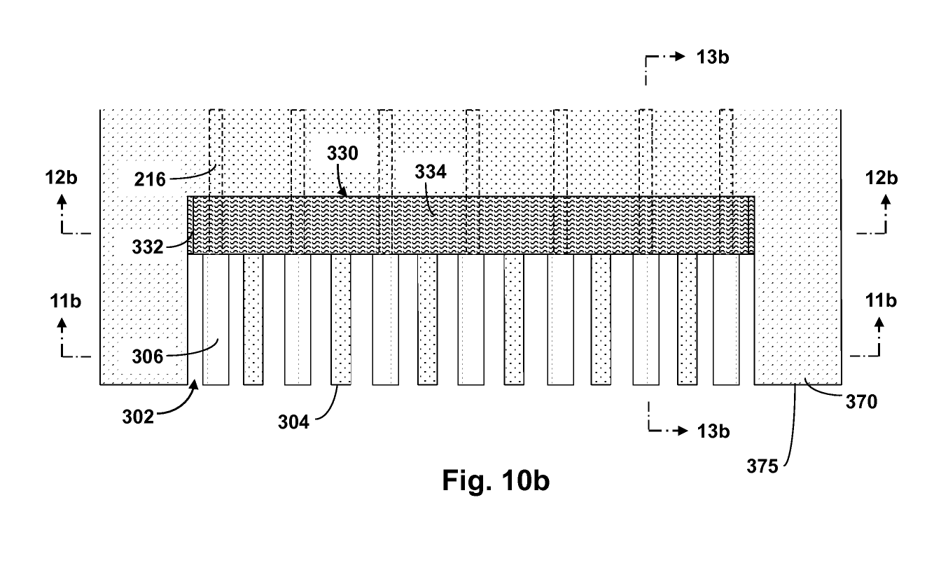

A conductive pad 306 can be disposed within each of the channels 302. The conductive pad 306 can be electrically coupled to the conductive trace 216. In some embodiments, the conductive pads 306 and the conductive traces 216 are formed of the same material. In such embodiments, the conductive pads 306 and the conductive traces 216 are separately described for clarity. In some embodiments, the conductive pads 306 and the conductive traces 216 include different materials and are described as distinct components. While FIGS. 3a, 4a, and 5a illustrate that the conductive traces 216 are provided on a surface of the conductor interface 220, it is understood that the conductive traces 216 can disposed within the conductor interface 220 (e.g., as illustrated in FIGS. 10a-10b and 12a-13b). The conductive pads 306 and the conductive traces 216 can facilitate electrical communication between the scanner assembly 106 and the cable 114, as well as other proximal components (such as the PIM coupler 122). For example, a conductor 218 can be soldered to the conductive pad 306 within each of the channels 302. The conductive pads 306 can be sized and shaped to physically contact the conductor 218 (e.g., directly or indirectly through solder). The dimensions of the conductive pads 306 (including, e.g., a height, a width, and a depth) can vary in different embodiments. For example, as illustrated in FIG. 5a, the width of the conductive pad 306 can be less than the width of the channel 302. In other embodiments, the width of the conductive pad 306 can be approximately equal to the width of the channel 302. For example, as illustrated in FIG. 4a, the conductive pads 306 can longitudinally extend along an entire depth of the channels 302. In other embodiments, the depth of the conductive pads 306 can be less than the depth of the channels 302.

While FIGS. 3a, 4a, and 5a illustrate that the conductor interface 220 includes seven channels 302, the number of channels can vary in different embodiments. The number of channels can be selected based on, e.g., the number of conductors 218 of the cable 114. For example, conductor interface 220 can include one, two, three, four, five, six, seven, or more channels. In some embodiments, the conductor interface 220 can include seven channels 302 when the cable 114 includes a seven-conductor transmission line. In some embodiments, the conductor interface 220 can include four channels 302 when the cable 114 includes a four-conductor transmission line. The dimensions (e.g., a height, a width, and a depth) of the channels 302 can vary in different embodiments. The dimensions of the channels 302 can selected at least partially based on the conductors 218. For example, one or more dimensions can be chosen to accommodate the gauge of the conductors 218, the number of conductors, etc. In some embodiments, the height 301 can be between about 0.0003 in and about 0.01 in, about 0.001 in and about 0.005 in, about 0.002 in and 0.004 in, and/or other suitable values. The width 303 can be between about 0.0003 in and about 0.01 in, about 0.001 in and about 0.005 in, about 0.002 in and 0.004 in, and/or other suitable values. The depth 305 can be between about 0.0003 in and about 0.01 in, about 0.001 in and about 0.01 in, about 0.001 in and 0.005 in, and/or other suitable values. It is understood that any suitable values for the dimensions can be selected such that electrical conductivity is maintained while the conductor interface 220 is being used. The conductor interface 220, with the suitable dimensions, can be designed and tested to ensure the IVUS device functions as desired during use. The channels 302 and the walls 304 can extend longitudinally along the conductor interface 220. In some embodiments, the channels 302 and the walls 304 have the same depth 305 such that the channels 302 are physically separated from one another. Thus, the conductors 218 of the cable 114 can be electrically isolated from one another when they are disposed within the channels 302.

Referring now to FIGS. 3b, 4b, and 5b, the conductor interface 220 includes a removable member 310. The removable member 310 can be described as a removable tab. The removable member 310 can be formed of a plastic material, a polymer material, a polyimide material, a polyester film, a polyimide film, a polyethylene napthalate film, a polyetherimide film, and/or other suitable materials. The removable member 310 can be disposed at least partially along a surface of the conductor interface 220. The removable member 310 can include a first portion 314 that is positioned in contact with the surface of the conductor interface 220 and a second portion 312 that extends away from the surface of the conductor interface 220. In the embodiment of FIGS. 3b, 4b, and 5b, the removable member 310 includes multiple first portions 314 that extend longitudinally along a top surface 221 of walls 304 of the conductor interface 220.

The removable member 310 can be removably coupled to the conductor interface 220. For example, an adhesive, such as a UV curable adhesive, cyanoacrylate adhesive, epoxy-based adhesive, and/or other suitable adhesives, can temporarily affix the removable member 310 to the conductor interface 220. In some embodiments, a mechanical coupling or electrostatic forces removably couple the removable member 310 to the conductor interface 220. In some embodiments, only the first portions 314 are removably coupled to the conductor interface 20. In some embodiments, the second portion 312 and the first portions 314 of the removable member 310 are removably coupled to the conductor interface 220.

The depth and width of the first portions 314 of the removable member 310 are selected to be approximately equal to or greater than the depth and width of top surfaces 221 of the walls 304 of the conductor interface 220. That is, the dimensions of the first portions 314 are selected such that the removable member 310 covers the top surface 221 of walls 304. When the conductors 218 are soldered within the channels 302, solder can flow along the top surface 221 of the walls 304 (e.g., when there is excess solder, when the solder is incorrectly directed, etc.). If the removable member 310 is not positioned along the top surface 221 of the walls 304, the solder on the top surface 221 of the walls 304 can facilitate unintentional and unwanted electrical communication between the conductors 218. When the removable member 310 is positioned along the top surface 221 of the walls 304, the excess solder flows along the first portions 314 (and not directly on the top surface 221 of the walls 304). The removable member 310 can be removed, along with the solder disposed on the first portions 314, to electrically isolate the conductors 218. Unintentional and unwanted electrical communication between the conductors 218 can be limited by removing the solder. For example, removing the removable member 310 can include includes pulling the second portion 312 such that the first portion 314 is no longer is contact with the surface of the conductor interface 220.

The channels 302 and the removable member 310 of the conductor interface 220 can facilitate more efficient manufacturing of the IVUS device. For example, rather than precisely solder the individual conductors 218 to corresponding conductive pads (a more laborious and time-intensive process), solder can be more freely and easily distributed along the top surface 221 of the conductor interface 220 (a less laborious and time-intensive process). The solder can flow into the individual channels 302 to electrically couple the conductors 218 and the conductive pads 306. The walls 304 electrically isolate the conductors 218 from one another. The solder that flows onto the removable member 310 can be removed when the removable member 310 is removed to further electrically isolate the conductors 218.

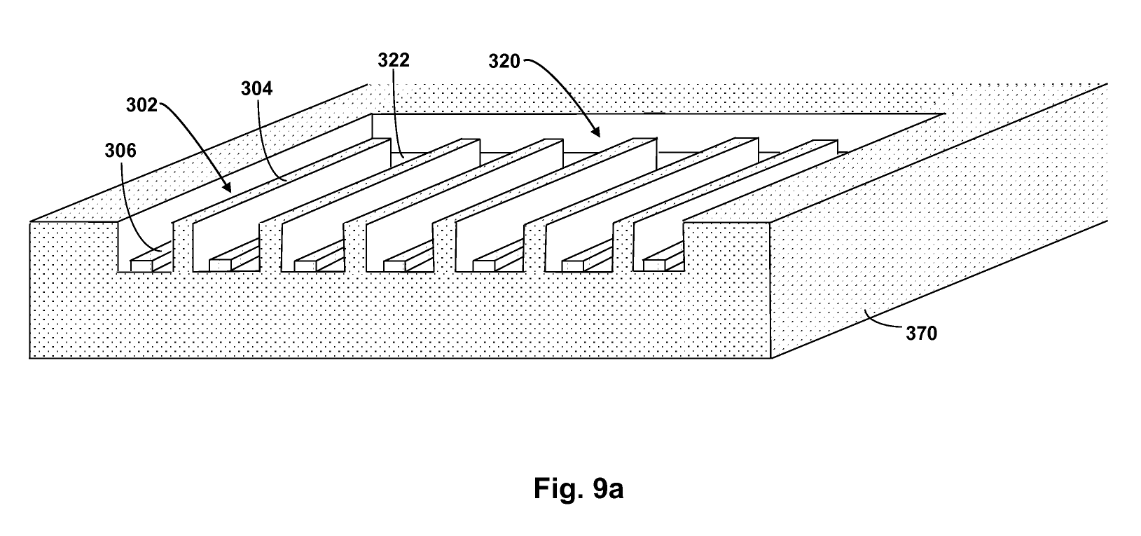

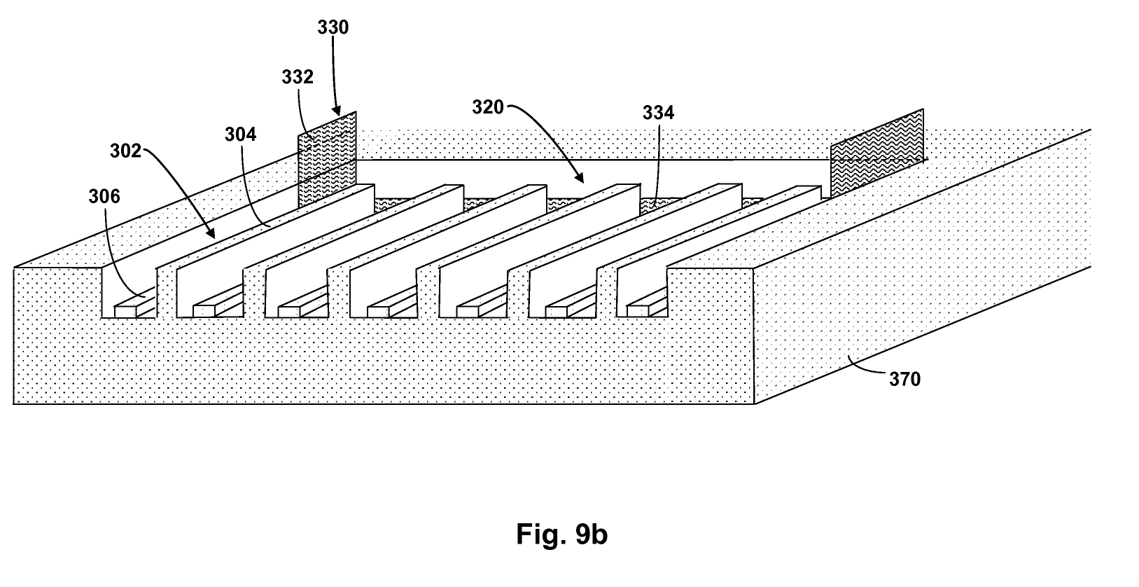

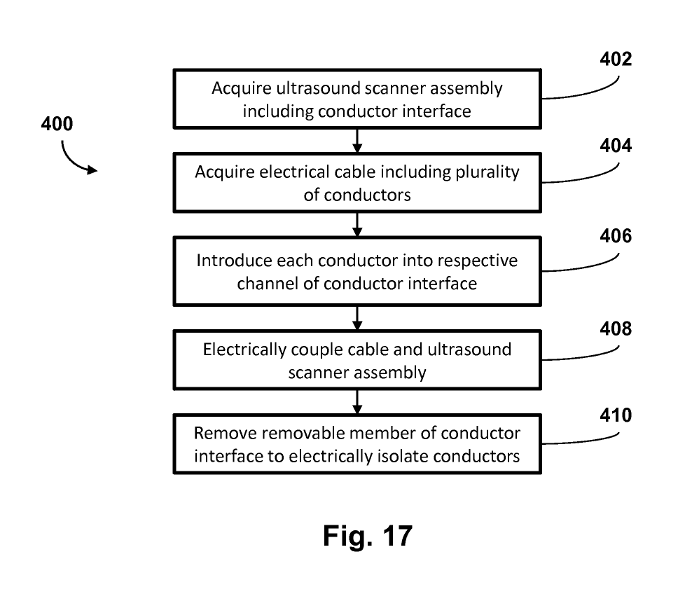

One exemplary embodiment of a method of manufacturing an IVUS device can be understood with reference to FIGS. 5b-8 and FIG. 17. FIGS. 5b-8 are cross-sectional front views of the conductor interface along the section line 5b-5b in FIG. 4b. FIG. 17 is a flow diagram of a method 400 for manufacturing an IVUS. Method 400 can be understood in the context multiple embodiments of the connector interface (e.g., the conductor interface 220 of FIGS. 3a-8, the conductor interface 370 of FIGS. 9a-16, etc.). The method 400 can include, at step 402, acquiring an ultrasound scanner assembly. For example, the ultrasound scanner assembly 106 can include ultrasound transducer array 202 disposed at a distal portion of the ultrasound scanner assembly. The ultrasound scanner assembly 106 can also include a transducer control circuit 206 electrically coupled to the ultrasound transducer array 202. The ultrasound scanner assembly 106 can include a conductor interface 220 disposed at a proximal portion of the ultrasound scanner assembly. The conductor interface 220 is illustrated in FIG. 5b. The conductor interface 220 includes a plurality of channels 302 and removable member 310 disposed at least partially along a surface of the conductor interface.

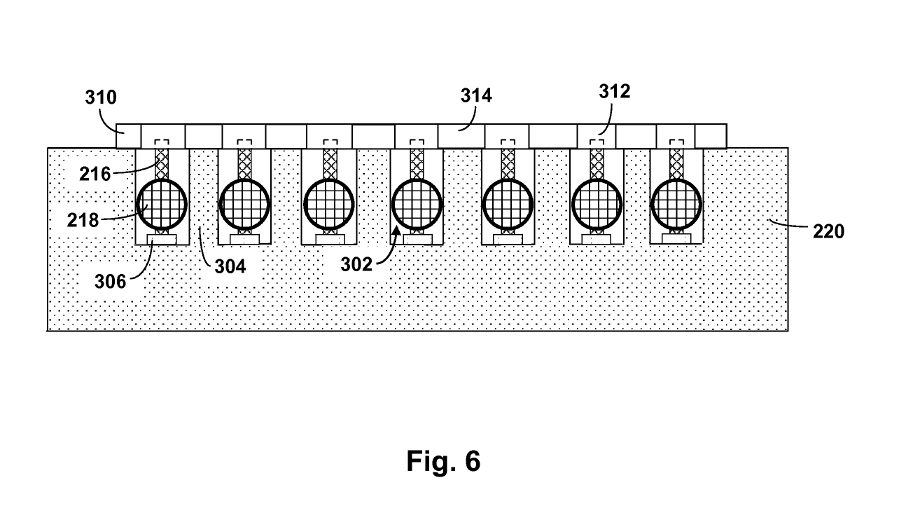

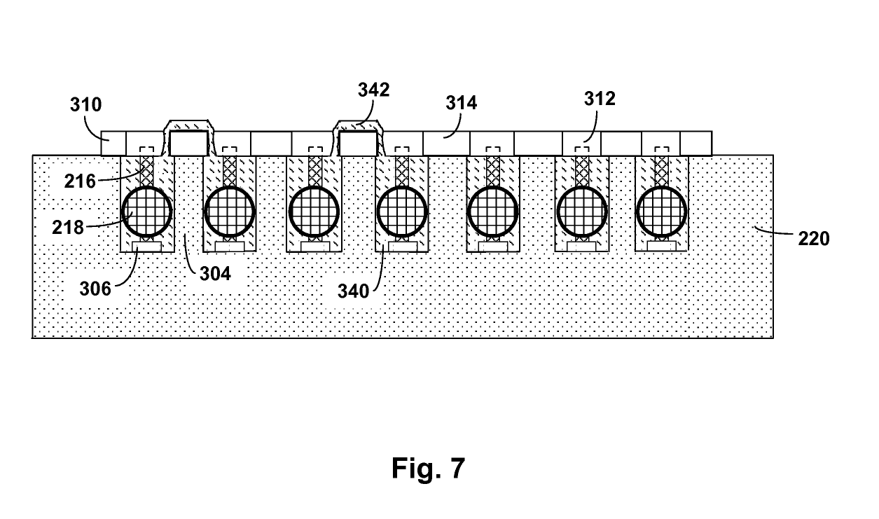

The method 400 can include, at step 404, acquiring an electrical cable including a plurality of conductors. The method 400 can also include, at step 406, introducing each conductor into a respective channel of the conductor interface. For example, as shown in FIG. 6, the conductors 218 of the cable 114 can be introduced into the channels 302 of the conductor interface 220. The method 400 can include, at step 408, electrically coupling the cable and ultrasound scanner assembly. In some embodiments, electrically coupling the cable and the ultrasound scanner assembly includes electrically coupling each of the plurality of conductors and a conductive pad disposed in each respective channel of the conductor interface. In some embodiments, electrically coupling each of the plurality of conductors and a conductive pad includes introducing solder into the respective channels of the conductor interface. For example, as shown in FIG. 7, the conductors 218 can be electrically coupled to the conductive pads 306 by the solder 340 disposed in the channels 302. The conductors 218 are electrically isolated from one another in that the conductors 218 are separated by the walls 304 of the conductor interface 220.

During step 408, excess solder 342 can become disposed on and/or around the removable member 310 (e.g., the first portions 314). Such circumstances can arise from human or machine error when solder is being introduced into the channels 302. Because of the small size of the channels 302, even a small error in the amount of solder used, in the location the solder is directed, etc., can cause excess solder 342 to accumulate in unintended locations of conductor interface 220. Excess solder 342 can create a path for electrical signals and cause inadvertent electrical communication between conductors 218. In conventional methods of assembling an IVUS device, the presence of excess solder can render the flex circuit and/or electrical cable unusable because there is no remedy for the unwanted electrical signal flow between conductors 218. As described below, the present disclosure advantageously provides a method of electrically isolating the conductors 218 even when excess solder 342 is present.

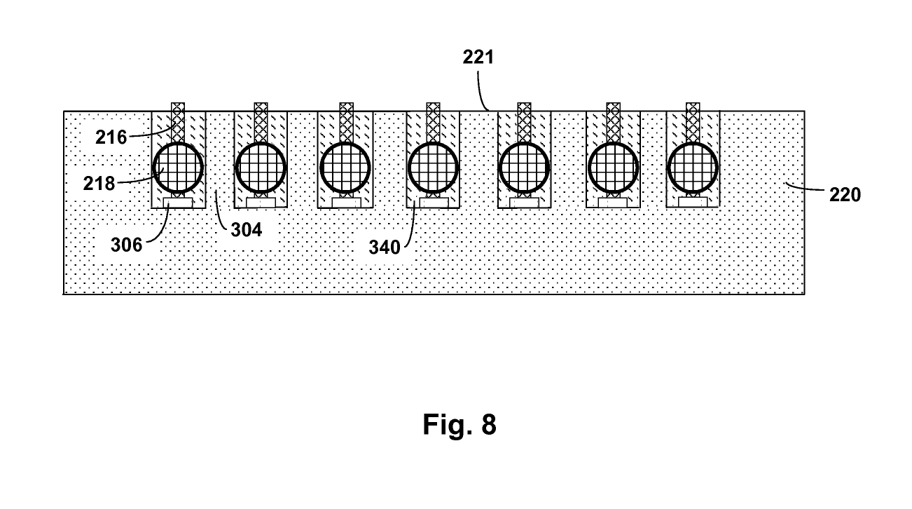

The method 400 can include, at step 410, removing a removable member of conductor interface to electrically isolate the conductors of the cable. For example, as seen in FIGS. 7 and 8, the removable member 310 can be removed from the conductor interface 220 after the conductors 218 are soldered to the conductive pads 306. Any excess solder 342 that is disposed on and/or around the removable member 310 after step 408 is removed when the removable member 310 is removed. Thus, the top surfaces 221 of the walls 304 do not include any solder. This can further electrically isolate the conductors 218 of the cable 114 by removing errant paths for electrical signal flow. In some embodiments, removing the removable member 310 includes pulling the second portion 312 such that the first portion 314 is no longer is contact with the surface of the conductor interface 220.

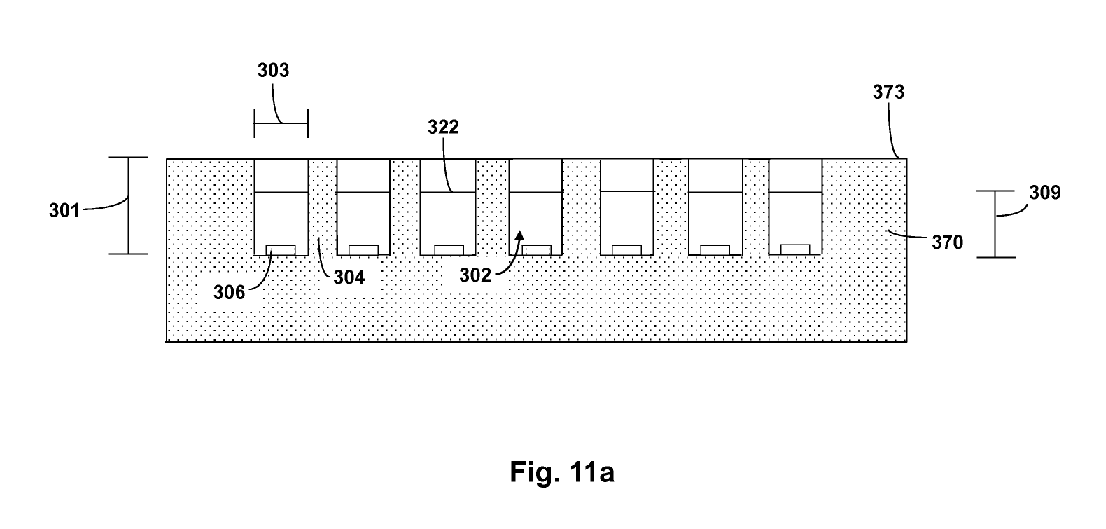

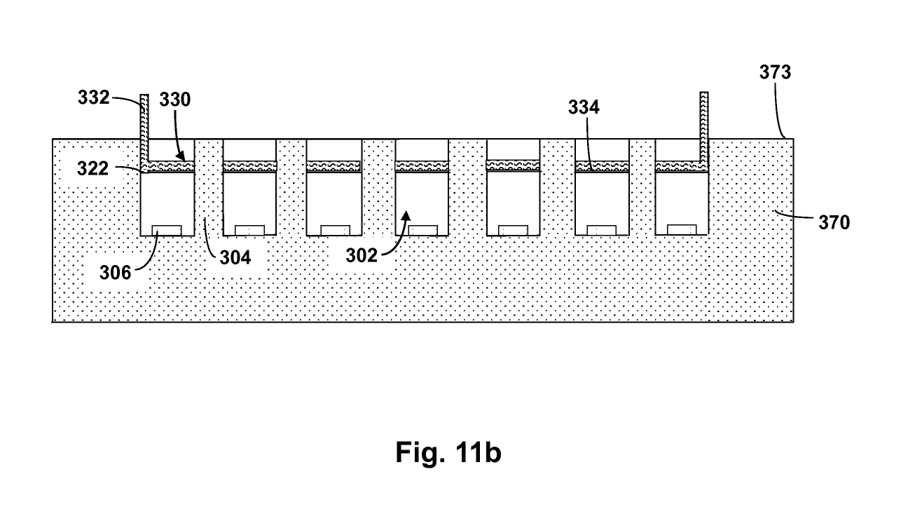

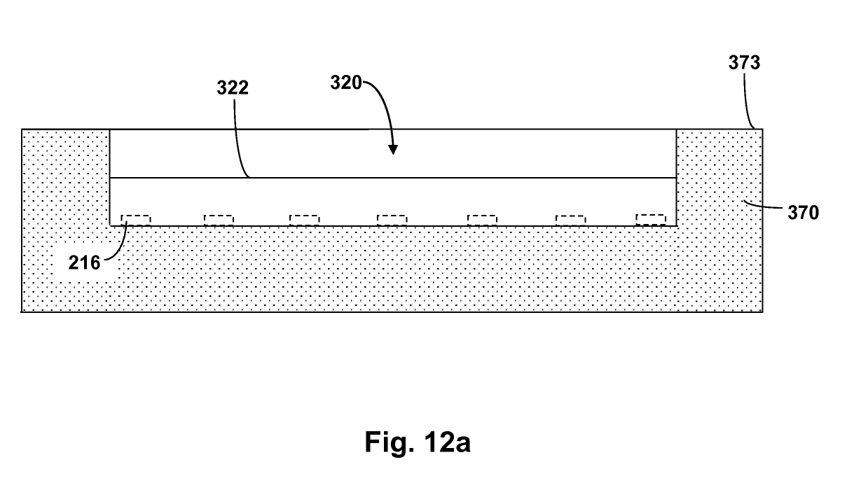

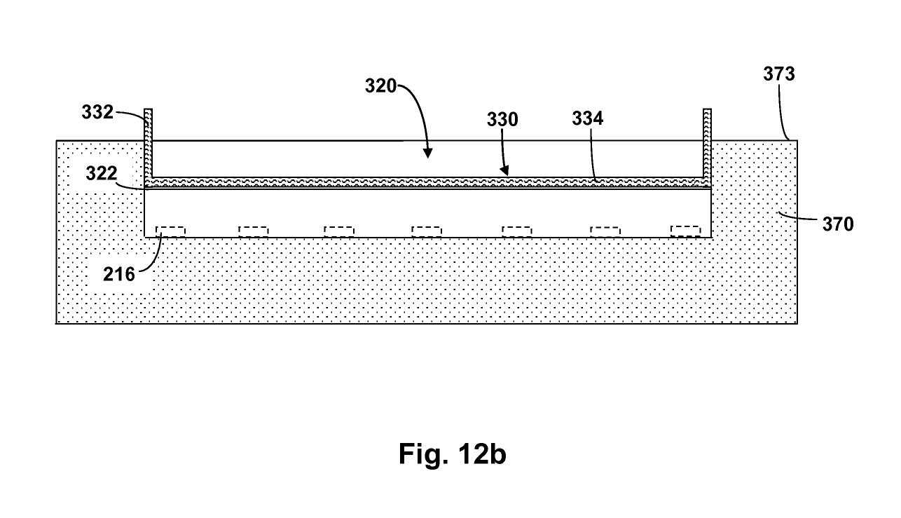

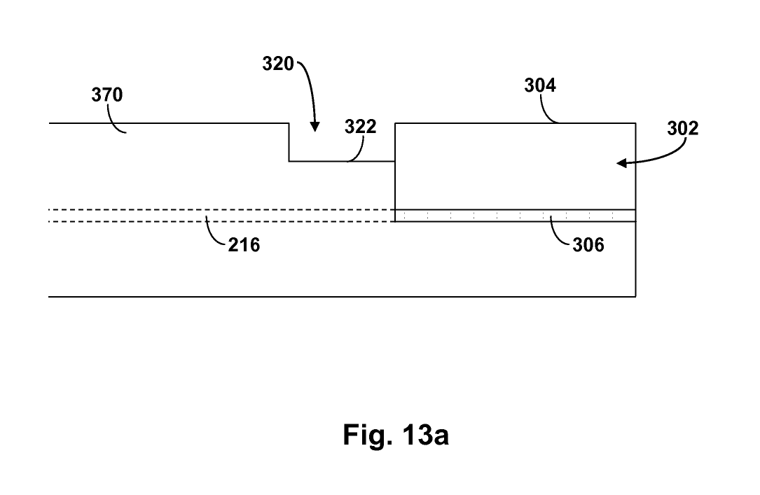

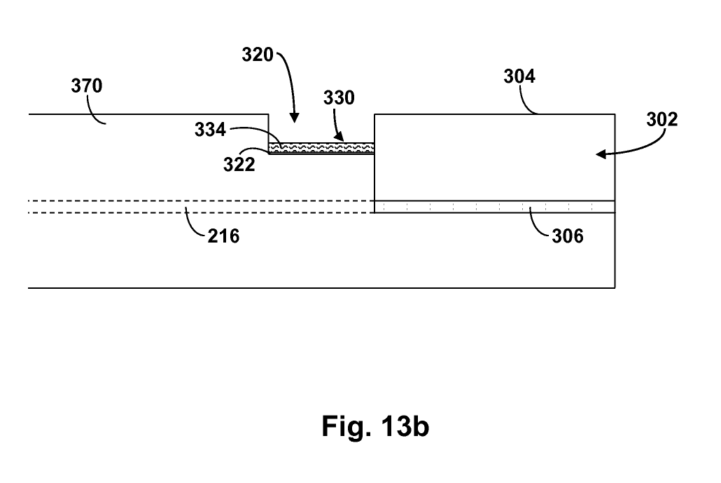

FIGS. 9a-13b illustrate an exemplary embodiment of the conductor interface 370 according to the present disclosure. FIGS. 9a and 9b are perspective views of the conductor interface 370. FIGS. 10a and 10b are top views of the conductor interface 370. FIGS. 11a and 12a are cross-sectional front views of the conductor interface 370 along the section lines indicated in FIG. 10a. FIGS. 11b and 12b are cross-sectional front views of the conductor interface 370 along the section lines indicated in FIG. 10b. FIGS. 13a and 13b are cross-sectional side views of the conductor interface 370 along the section lines indicated in FIGS. 10 and 10b, respectively. The conductor interface 370 of FIGS. 9b, 10b, 11b, 12b, and 13b includes a removable member 330, while the removable member 330 is omitted in the FIGS. 9a, 10a, 11a, 12a, and 13a.

The conductor interface 370 of FIGS. 9a-13b is similar in many respects to the conductor interface 220 of FIGS. 3a-5b. For example, the conductor interface 370 includes the channels 302 defined by the walls 304. The channels 302 can have a height 301 (FIG. 11a), a width 303 (FIG. 11a), and a depth 325 (FIG. 10a). As described with respect to FIGS. 3a-5b, one or more dimensions of the channels 302 can be selected at least partially based on the dimensions of the conductors 218 of the cable 114. The conductor interface 370 includes the conductive pads 306 disposed within the channels 302. The conductive pads 306 are in electrical communication with the conductive traces 216, which are disposed within the conducer interface 370 (as illustrated in, e.g., FIGS. 10a-10b and 12a-13b).

Referring to FIGS. 9a, 10a, 11a, 12a, and 13a, the conductor interface 370 includes a recessed shelf 322. The recessed shelf 322 is positioned distal of the channels 302. The recessed shelf 322 is disposed at a height 309 (FIG. 11a) such that the recessed shelf 322 is below the top surface 373 of the conductor interface 370. The recessed shelf 322 extends transversely across the conductor interface 370. The recessed shelf 322 can extend at least across all of the channels 302 and the walls 304. That is, the width of the recessed shelf 322 can be equal to or greater than the total width of the channels 302 and the walls 304.

The depth 325 (FIG. 10a) of the channels 302 in FIGS. 9a-13b can include a longitudinal extent spanned by the walls 304 and the recessed shelf 322. The depth 327 (FIG. 10a) of the walls 304 in FIGS. 9a-13b can extend from a front surface 375 of the conductor interface 370 to the recessed shelf 322. Thus, the channels 302 are physically separated from one another by the walls 304 along the depth 327. The connector interface 370 can include a shared filling channel 320. The filling channel 320 can be at least partially defined by the recessed shelf 322. For example, the filling channel 320 can be disposed within the conductor interface 370 in the space above the recessed shelf 322 such that the recessed shelf 322 defines a bottom surface of the filling channel 320. The channels 302 can be in fluid communication via the recessed shelf 322 and the filling channel 320. As described below, the fluid communication between the channels 302 permitted by the recessed shelf 322 and the filling channel 320 can be advantageously utilized to efficiently solder the conductors 218 to the conductive pads 306 during manufacture of the IVUS device.

Referring now to FIGS. 9b, 10b, 11b, 12b, and 13b, the conductor interface 370 includes a removable member 330. The removable member 330 is similar in many respects to the removable member 310 of FIGS. 3b, 4, and 5b. For example, the removable member 330 can be formed of one or more of the materials described with respect to removable member 310. The removable member 330 can be disposed at least partially along a surface of the conductor interface 370. The removable member 310 can include a first portion 334 that is positioned in contact with the surface of the conductor interface 370 and a second portion 332 that extends away from the surface of the conductor interface 370. The first portion 334 and/or the second portion 332 can be removably coupled to the conductor interface 370.

In the embodiment of FIGS. 9b, 10b, 11b, 12b, and 13b, the first portion 334 of the removable member 300 is transversely disposed along a surface of the recessed shelf 322 of the conductor interface 220. The removable member 330 can be described as being positioned along the bottom surface of the filing channel 320. The depth and width of the first portion 334 is selected to be approximately equal to or greater than the depth and width of the recessed shelf 322. That is, the dimensions of the first portion 334 are selected such that the removable member 330 covers the surface of the recessed shelf 322. When the conductors 218 are soldered within the channels 302, solder can be introduced into the filling channel 320 and onto the recessed shelf 322. The solder can then flow from the recessed shelf 322 into the channels 302. If the removable member 330 is not positioned along the surface of the recessed shelf 322, the solder on the recessed shelf 322 can facilitate unintentional and unwanted electrical communication between the conductors 218. When the removable member 330 is positioned along the surface of the recessed shelf 322, the solder flows along the first portion 334 (and not directly on the surface of the recessed shelf 322). The removable member 330 can be removed, along with the solder disposed on the first portions 334, to electrically isolate the conductors 218. Unintentional and unwanted electrical communication between the conductors 218 can be limited by removing the solder. For example, removing the removable member 330 can include pulling the second portion 332 such that the first portion 334 is no longer is contact with the surface of the conductor interface 220. The removable member 330 can include two second portions 332. The removable member 330 can be removed by pulling both simultaneously or either one of the second portions 332.

The channels 302, the filling channel 320, the recessed shelf 322, and the removable member 330 of the conductor interface 370 can facilitate more efficient manufacturing of the IVUS device. For example, rather than precisely solder the individual conductors 218 to corresponding conductive pads (a more laborious and time-intensive process), solder can be more freely and easily distributed into the filling channel 320 and onto the recessed shelf 322, which is covered by the removable member 330. The solder can then flow into the individual channels 302 to electrically couple the conductors 218 and the conductive pads 306. The walls 304 can at least partially electrically isolate the conductors 218 from one another. The solder that flows onto the removable member 330 can be removed when the removable member 330 is removed to further electrically isolate the conductors 218.

One exemplary embodiment of a method of manufacturing an IVUS device can be understood with reference to FIGS. 13b-17. FIGS. 13b-16 are cross-sectional side views of the conductor interface along the section line 13b-13b in FIG. 10b. As described above, FIG. 17 is a flow diagram of a method 400 for manufacturing an IVUS device. Method 400 can be understood in the context multiple embodiments of the connector interface (e.g., the conductor interface 220 of FIGS. 3a-8, the conductor interface 370 of FIGS. 9a-16, etc.). The method 400 can include, at step 402, acquiring an ultrasound scanner assembly. As described above, the ultrasound scanner assembly 106 can include ultrasound transducer array 202, a transducer control circuit 206, and a conductor interface 370. The conductor interface 220 is at least partially illustrated in FIG. 13b. The conductor interface 370 includes a plurality of channels 302, a filling channel 320, and a removable member 330 disposed at least partially along a surface of the conductor interface.

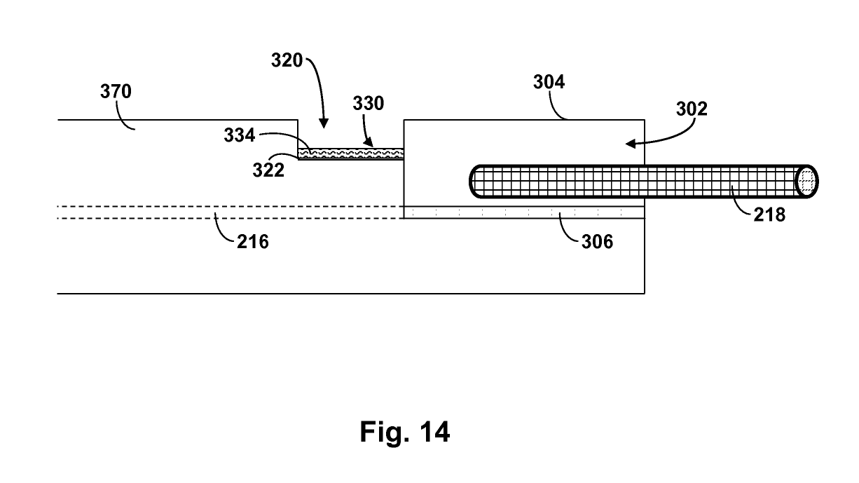

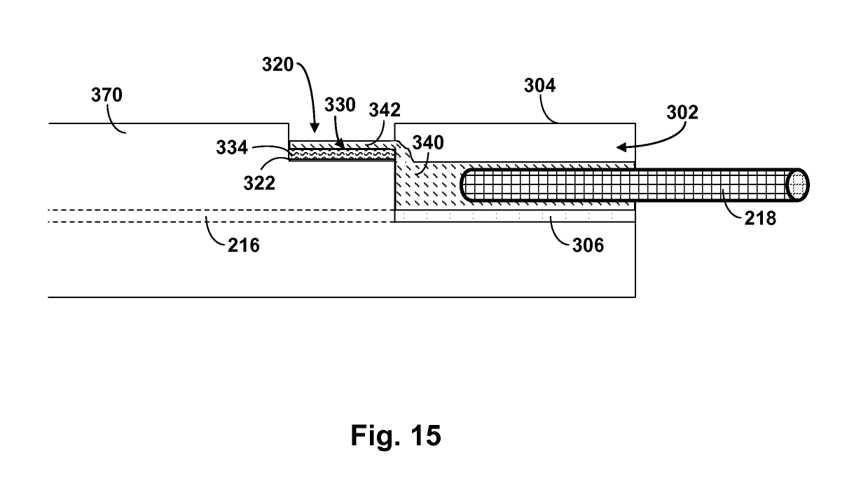

The method 400 can include, at step 404, acquiring an electrical cable including a plurality of conductors. The method 400 can also include, at step 406, introducing each conductor into a respective channel of the conductor interface. For example, as shown in FIG. 14, the conductor 218 of the cable 114 can be introduced into the channel 302 of the conductor interface 370. The method 400 can include, at step 408, electrically coupling the cable and ultrasound scanner assembly. As described above, step 408 can include electrically coupling each of the plurality of conductors and a conductive pad disposed in each respective channel of the conductor interface and/or introducing solder into the respective channels of the conductor interface. For example, as shown in FIG. 15, the conductors 218 can be electrically coupled to the conductive pads 306 by the solder 340 disposed in the channel 302. The solder 340 can be first introduced into the filling channel 320 and onto the first portion 334 of the removable member 330. The solder 340 can then flow from the first portion 334 into the channels 302 and into contact with the conductors 218 and the conductive pads 306. The conductors 218 can be at least partially electrically isolated from one another in that the conductors 218 are separated by the walls 304 of the conductor interface 370.

During step 408, excess solder 342 can become disposed on removable member 330 (e.g., the first portion 334). In the embodiment of FIGS. 9a-16, solder is purposely introduced onto the removable member 330 such that the solder flows into the channels 302 as an efficient method of electrically coupling the conductors 218 and the conductive pads 306. The excess solder 342, however, creates an unwanted electrical signal flow path between conductors 218. If the excess solder 342 remains on the conductor interface 370, errant electrical communication between arise between conductors 218. As described below, the present disclosure advantageously provides a method of electrically isolating the conductors 218 when excess solder 342 is present.

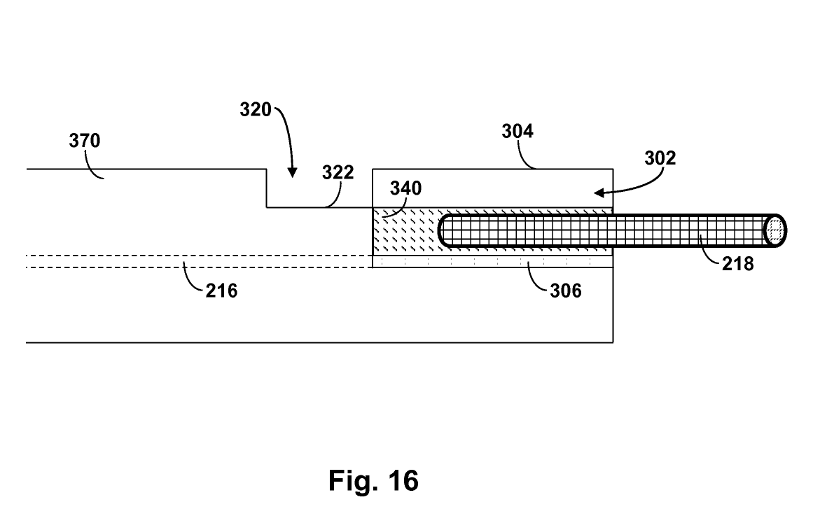

The method 400 can include, at step 410, removing a removable member of conductor interface to electrically isolate the conductors of the cable. For example, as seen in FIGS. 15 and 16, the removable member 330 can be removed from the conductor interface 370 after the conductors 218 are soldered to the conductive pads 306. Any excess solder 342 that is disposed on the removable member 330 after step 408 is removed when the removable member 310 is removed. Thus, the recessed shelf 322 does not include any solder. This can further electrically isolate the conductors 218 of the cable 114 by removing errant paths for electrical signal flow. Thus, even though the channels 302 are in fluid communication via the filling channel 320 and the recessed shelf 322, removing the removable member 330 can electrically isolate the conductors 218 positioned within the channels 302. In some embodiments, removing the removable member 330 includes pulling the second portion 332 such that the first portion 334 is no longer is contact with the surface of the conductor interface 370.

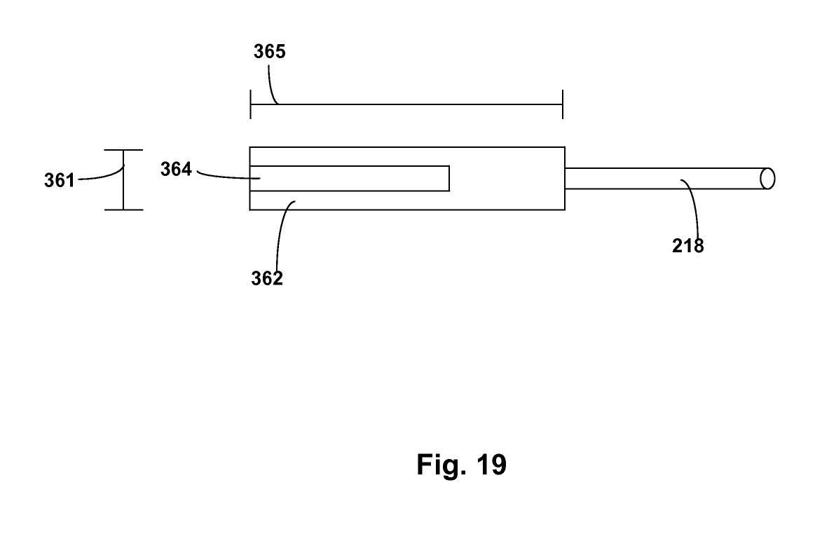

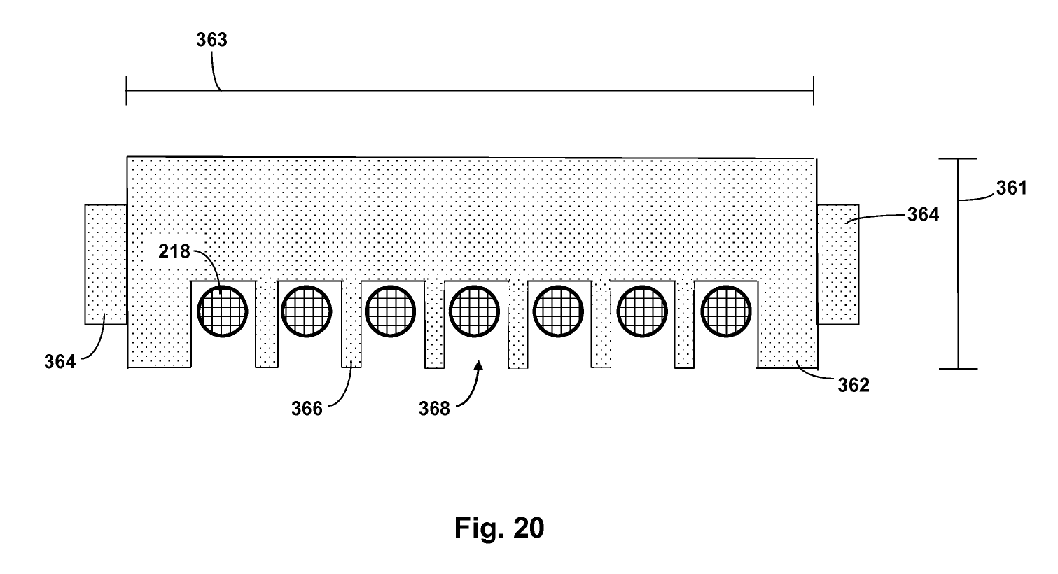

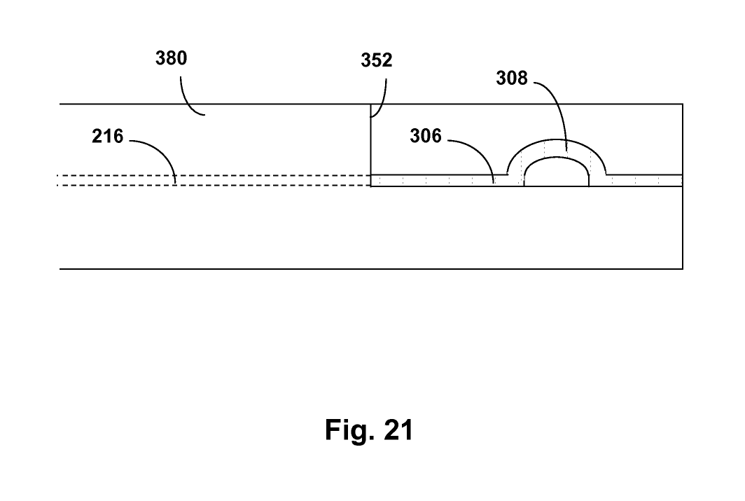

FIGS. 18-21 illustrate an exemplary embodiment of a conductor interface 380 and a distal connector 362 according to the present disclosure. FIG. 18 is a top view of the conductor interface 380 and the distal connector 362 of the cable 114. FIG. 19 is a side view of the distal connector 362. FIG. 20 is cross-sectional front view of the distal connector 362 along the section line indicated in FIG. 18. FIG. 21 is a cross-sectional side view of the conductor interface 380 along the section line indicated in FIG. 18.

The electrical cable 114 can include a connector 362 at a distal portion. The distal connector 362 can be formed of a plastic, thermoplastic, polymer, hard polymer, carbon fiber, and/or other suitable material(s). The dimensions (e.g., a height, a width, and a depth) of the distal connector 362 can vary in different embodiments. The dimensions of the distal connector 362 can be selected at least partially based on the conductors 218. For example, one or more dimensions can be chosen to accommodate the gauge of the conductors 218, the number of conductors, etc. In some embodiments, the height 361 can be between about 0.001 in and about 0.015 in, about 0.001 in and about 0.012 in, about 0.001 and about 0.01 in, and/or other suitable values. The width 363 can be between about 0.001 in and about 0.02 in, about 0.001 in and about 0.015 in, about 0.001 in and about 0.01 in, and/or other suitable values. The depth 365 can be between about 0.001 in and about 0.02 in, about 0.001 in and about 0.015 in, about 0.001 in and about 0.01 in, and/or other suitable values. It is understood that any suitable values for the dimensions can be selected such that electrical conductivity is maintained while the distal connector 362 is being used. The distal connector 362, with the suitable dimensions, can be designed and tested to ensure the IVUS device functions as desired during use.

The distal connector 362 can include a plurality of channels 368 (FIG. 20). A distal portion of each of the conductors 218 of the cable 114 can be received in a respective channel 368. The conductors 218 can be coupled to the distal connector 362 at the channels 368 via a suitable adhesive and/or a mechanical coupling. The channels 368 and the conductors 218 can be separated and electrically isolated from one another by walls 366 of the distal connector 362. For example, the walls 366 can extend past the conductors 218 to ensure than the conductors are electrically isolated from one another. The number and dimensions (e.g., a height, a width, and a depth) of the channels can vary in different embodiments. The dimensions of the channels 368 can be selected at least partially based on the dimensions of the conductors 218.

The distal connector 362 is configured to mechanically engage the conductor interface 380 to electrically couple, e.g., the PIM coupler 122 at a proximal portion of the cable 114 and the ultrasound scanner assembly 106. Thus, the conductor interface 380 includes a channel 352 sized and shaped to receive the distal connector 362. The distal connector 362 can include a flexible locking member 364 extending transversely from the connector. In some embodiments, a flexible locking member 364 is provided on each side of the distal connector 362. The locking members 364 are configured to flex inwards towards the sides of the distal connector 362 as the connector passes a narrow portion when introduced into the conductor interface 380. The conductor interface 380 can include corresponding locking channels 354 sized and shaped to receive the locking members 364 of the distal connector 362. The locking members 364 are configured to flex outwards after passing the narrow portion of the conductor interface 380 such that the locking members 364 are received in the locking channels 354. Once the distal connector 362 is completely received within the channel 352 and the locking members 364 are completely received with in the locking channels 354, the cable 114 can be fixedly secured and electrically coupled to the scanner assembly 106.

The conductor interface 380 also includes conductive pads 306 and conductive traces 216, similar to the conductive pads 206 and conductive traces 216 described herein. The scanner assembly 106 can be electrically coupled to the PIM coupler 122 when the conductors 218 of the cable 114 are electrically coupled to the conductive pads 306. In some embodiments, the conductive pads 306 are entirely planar along the conductor interface 380. In some embodiments, as illustrated in FIG. 21, at least a portion of the conductive pads 306 is curved. The curved portions 308 can physically contact the conductors 218 disposed within the distal connector 362. The combination of the curved portions 308 and the walls 366 of the distal connector 362 can ensure than there is no inadvertent electrical signal flow between conductive pads 306 and/or conductors 218. Because the walls 366 extend beyond the conductors 218 in the distal connector 362, the curved portion 308 extends upwards to make contact with the conductors 218. Thus, contact between the curved portions 308 and the conductors 218 occurs at locations that are higher than the other portions of the conductive pads 306. The locations at which contact occurs are electrically isolated from one another by the walls 366 of the distal connector 362.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. For example, the features of various embodiments can be combined with features of different embodiments. One or more steps can be added to or removed from the methods described herein. A person of ordinary skill in the art will understand that the steps of the method can be performed in an order different than the order described herein. It is understood that such variations may be made to the foregoing without departing from the scope of the present disclosure. Accordingly, it is appropriate that the appended claims be construed broadly and in a manner consistent with the present disclosure.

While the present disclosure referred to primarily to intravascular devices, the system disclosed herein is well suited to any medical device including an electrical coupling between conductors of a cable and another component. For example, the teachings of the present disclosure can be implemented in transluminal and/or endoscopic devices. One skilled in the art will recognize the application of the principles herein across other disciplines.

What is claimed is:

1. A method of manufacturing an intravascular ultrasound (IVUS) device, comprising:
acquiring an ultrasound scanner assembly, the ultrasound scanner assembly including:
an ultrasound transducer array disposed at a distal portion of the ultrasound scanner assembly;
a transducer control circuit electrically coupled to the ultrasound transducer array; and
a conductor interface disposed at a proximal portion of the ultrasound scanner assembly, wherein the conductor interface includes a plurality of channels and a removable member disposed at least partially along a surface of the conductor interface;
acquiring a cable including a plurality of conductors;
introducing each of a plurality of conductors into a respective channel of the conductor interface;
electrically coupling the cable and the ultrasound scanner assembly; and
removing the removable member from the conductor interface to electrically isolate the conductors of the cable from one another.

2. The method of claim 1, wherein electrically coupling the cable and the ultrasound scanner assembly includes electrically coupling each of the plurality of conductors and a conductive pad disposed in each respective channel of the conductor interface.

3. The method of claim 2, wherein electrically coupling each of the plurality of conductors and a conductive pad includes introducing solder into the respective channels of the conductor interface.

4. The method of claim 1, wherein the removable member comprises a first portion positioned in contact with the surface of the conductor interface and a second portion extending away from the surface of the conductor interface.

5. The method of claim 1, wherein removing the removable member includes pulling the second portion of removable member such that the first portion of the removable member is no longer is contact with the surface of the conductor interface.

6. The method of claim 1, wherein the removable member comprises a plurality of first portions longitudinally disposed along a top surface of walls of the conductor interface, the walls defining the plurality of channels therebetween.

7. The method of claim 1, wherein the conductor interface further includes a recessed shelf disposed distal of the plurality of channels, and wherein the first portion of the removable member is transversely disposed along a surface of the recessed shelf.

8. The method of claim 7, wherein electrically coupling each of the plurality of conductors and a conductive pad includes introducing solder onto the recessed shelf such that the solder flows into each respective channel of the conductor interface.

9. The method of claim 7, wherein electrically coupling each of the plurality of conductors and a conductive pad includes introducing solder into a filling channel at least partially defined by the recessed shelf such that the solder flows into each respective channel of the conductor interface, the filling channel being in fluid communication each respective channel.

10. The method of claim 1, wherein the ultrasound scanner assembly comprises a flex circuit such that the ultrasound transducer array and the transducer control circuit are electrically and physically coupled to the flex circuit.

11. An ultrasound scanner assembly, comprising:
an ultrasound transducer array disposed at a distal portion of the ultrasound scanner assembly;
a transducer control circuit electrically coupled to the ultrasound transducer array; and
a conductor interface disposed at a proximal portion of the ultrasound scanner assembly, wherein the conductor interface includes a plurality of channels and a removable member disposed at least partially along a surface of the conductor interface.

12. The ultrasound scanner assembly of claim 11, wherein the conductor interface further comprises a conductive pad disposed in each of the plurality of channels.

13. The ultrasound scanner assembly of claim 11, wherein the removable member comprises a first portion positioned in contact with the surface of the conductor interface and a second portion extending away from the surface of the conductor interface.

14. The ultrasound scanner assembly of claim 13, wherein the removable member comprises a plurality of first portions longitudinally disposed along a top surface of walls of the conductor interface, the walls defining the plurality of channels therebetween.

15. The ultrasound scanner assembly of claim 13, wherein the conductor interface further includes a recessed shelf disposed distal of the plurality of channels, and wherein the first portion of the removable member is transversely disposed along a surface of the recessed shelf.

16. The ultrasound scanner assembly of claim 15, wherein the conductor interface further includes a filling channel at least partially defined by the recessed shelf and disposed in the space above the recessed shelf.

17. The ultrasound scanner assembly of claim 13, wherein ultrasound scanner assembly comprises a flex circuit such that the ultrasound transducer array and the transducer control circuit are electrically and physically coupled to the flex circuit.

18. An intravascular ultrasound (IVUS) device, comprising:
a flexible elongate member;
a patient interface module (PIM) coupler disposed at a proximal portion of the flexible elongate member;
an ultrasound scanner assembly disposed at a distal portion of the flexible elongate member, the ultrasound scanner assembly including:
an ultrasound transducer array disposed at a distal portion of the ultrasound scanner assembly;
a transducer control circuit electrically coupled to the ultrasound transducer array; and
a conductor interface disposed at a proximal portion; and
a cable disposed within and extending along a length of the flexible elongate member between the ultrasound scanner assembly and the interface coupler, the cable including a plurality of conductors, the cable further including a connector at a distal portion configured to mechanically engage the conductor interface to electrically couple the interface coupler and the ultrasound scanner assembly.

19. The intravascular device of claim 18, wherein the flexible elongate member includes at least one of a catheter, a guide wire, or a guide catheter.
